(19) United States
(12) Patent Application Publication
(10) Pub. No.: US 2012/0329989 A1
(43) Pub. Date: Dec. 27, 2012

(54) METHOD FOR PRODUCING A GRAPHITE-BASED PEPTIDE PURIFICATION MATERIAL AND METHOD FOR PEPTIDE PURIFICATION

(75) Inventor: Oliver J. Kreuzer, Berlin (DE)

(73) Assignee: PEPTIDES & ELEPHANTS GMBH, Berlin (DE)

(21) Appl. No.: 13/586,196

(22) Filed: Aug. 15, 2012

(30) Foreign Application Priority Data
Feb. 16, 2010 (DE) 102010001983.6
Feb. 15, 2011 (EP) PCT/EP2011/052214

(51) Int. Cl. C07K 1/30; C01B 31/00; C01B 31/04

(52) U.S. Cl. 530/344; 423/448

(57) ABSTRACT

The invention relates to a method for producing a graphite-based peptide purification material, which is characterized in that graphite is adjusted to a pH of <7 (acid) by incubation at least once in at least one organic or inorganic acid for at least one minute. The invention further relates to a method for peptide purification, wherein the peptide has a terminal planar aromatic protective group, using graphite in a packed form as the purification material, wherein the method is characterized in that previously acidified graphite (pH<7), which has been produced according to the method for producing a graphite-based peptide purification material, is used as the purification material.

The present invention relates to a method for producing a graphite-based peptide purification material and a method for peptide purification in which the graphite-based peptide purification material is used.

In peptide synthesis, the amino acids are linked to each other in the order of the peptide sequence. The peptide chain grows by one amino acid per synthesis step and in solid phase synthesis it is attached to a solid carrier. One end of each amino acid, the C-terminus, is coupled to the other end of the preceding amino acid, the N-terminus. Both termini are reactive, which means that during peptide synthesis it must be ensured that the amino acid does not react with itself, or in the case of multiple amino acids, that they do not become linked in the wrong order. In order to introduce selectivity, therefore, the carboxyl and amino groups that are not to be linked are provided with a protecting group.

In solid-phase synthesis, first the C-terminus of an amino acid that is protected at the N-terminus is attached to the surface of the solid phase. To do this, the N-terminus of the amino acid that is to be added is blocked with a temporary protecting group. This protecting group must be removed before each new synthesis step. The functional groups of the amino acid side chains are blocked throughout the entire synthesis process by “permanent” protecting groups. At the end of a synthesis, the amino acid that was introduced first is detached from the substrate and protonised to obtain the completed peptide. Finally, a free peptide then remains in a cleaving solution.

The completed peptide is detached from the carrier by using a strong acid, preferably trifluoroacetic acid (TFA). In this process, the permanent protecting groups of the peptide side chains are cleaved at the same time. The temporary protecting group on the N-terminus is normally cleaved with a base. The free peptide and the cleaved protecting groups of the side chains are then present in the TFA solution.

In order to separate the peptide from the solution, it is common to implement an ether precipitation process. The addition of ether causes the peptide to precipitate, while the side chain protecting groups remain in the solution.

The purity of the peptides obtained by solid-phase synthesis is extremely variable. Not all amino acids can be linked completely. Also, a number of false sequences are created as well as the desired target sequence. These false sequences are not separated during ether precipitation.

The consequence of this is that one or more additional purification steps are needed in order to obtain the pure peptide. Depending on the length of peptide and its physico-chemical properties, as well of those of the byproducts, this separation can be extremely difficult. For example, unwanted byproducts whose length is only slightly different from that of the desired peptide may also have practically the same physico-chemical characteristics. The situation is further complicated by the fact that while synthesis on the solid phase can be carried out at a high production rate, the subsequent purification operation in its present form is nowhere near as efficient. The peptide is usually purified using high-performance liquid chromatography (HPLC) in a preparative HPLC system.

Such an HPLC system is expensive and is capable of purifying approximately a single peptide per hour. To this must be added the further costs for purchasing and disposing of the solvents used in liquid chromatography.

Another disadvantage is that the method allows of limited parallel operation, because only one peptide can be purified on an HPLC system. In order to purify multiple peptides in parallel, additional HPLC systems must be used, and this entails corresponding additional costs.

In international patent application WO 2005/118617 A2, Jacob et al. describe a purification method in which the protecting group of the N-terminus is not cleaved, but instead is used for the purification. The peptide-with-protecting group is transferred to a lipophilic surface, to which the protecting group binds. Then, non-bound impurities are washed off. This is followed by a washing step in which the binding between the peptide and the protecting group is broken. As a result, the protecting group remains on the lipophilic material and the free peptide is washed off. Various substances, for example reverse-phase materials, are used as the lipophilic materials.

This purification method has the disadvantage that the peptide is cleaved from the protecting group while purification, is actually in progress, and this may lead to secondary reactions. Also, it can sometimes be advantageous to keep the protecting group initially. Some of the lipophilic materials Jacob et al. use are also extremely expensive.

A less expensive and readily obtainable purification material would be graphite. Graphite has already been studied by Ramage and Raphy, at which time various planar aromatic systems were used as the protecting group (Tetrahedron Letters, 33, 3, 385-388, 1392). In this case, however, the behaviour of the individual protecting groups proved to be highly inconsistent. Particularly the fluorenylmethoxycarbonyl group (Fmoc group), which was expected to provide good support on the graphite, proved to be unsuitable. In the work by Ramage and Raphy, only the tetrabenzofluorenylmethoxycarbonyl group (Tbfmoc group) was found to bond adequately with the graphite.

The object of the invention was to provide a graphite material that overcomes the disadvantages described in the preceding. A further object was to suggest a method based on this material with which peptides with a terminal planar aromatic protecting group may be purified.

This object is solved with a method for producing a graphite-based peptide purification material according to claim 1 and a method for purifying peptides according to claim 6. Other preferred embodiments are described in the dependent claims.

In other words, the object is solved with a method for producing a graphite-based peptide purification material that is characterised in that graphite is adjusted to a pH<7 (acid) by incubation at least once in at least one organic or inorganic acid for at least one minute. The object is further solved by a method for peptide purification wherein the peptide has a terminal planar aromatic protecting group, wherein this method using graphite in packed form is characterised in that the previously acidified graphite (pH<7) is used as the purification material.

The term peptide or peptides is understood to mean polypeptides having 2 to 100 amino acids, preferably having 2 to 40 amino acids, most preferably having 2 to 20 amino acids.

Planar aromatic protecting groups contain all corresponding protecting groups that are used in peptide synthesis. These include in particular fluorenylmethoxycarbonyl groups (Fmoc groups), tetrabenzofluorenylmethoxycarbonyl groups (Tbfmoc groups) and benzyloxycarbonyl groups (Z groups) and derivatives thereof with annelated benzene rings. Use of an Fmoc group is particularly preferred.

These temporary protecting groups, particularly the Fmoc group, are used in solid phase synthesis.

After each coupling step, all false sequences that have not reacted with the next amino acid are acetylated, and are blocked thereby. Only the completely synthesized target peptide still has the temporary protection group at the end of the synthesis.

Then, a solid phase extraction is carried out with a column material tuned specifically to the protecting group, as a result of which the protected target peptide is obtained from the TFA solution without ether precipitation. This also separates the false sequences at the same time. The protecting group at the N-terminus is retained.

The requisite column material is preferably manufactured by a method for producing a graphite-based peptide purification material in which the graphite is acidified in consecutive incubations in at least two acids for at least two minutes each time, wherein it is treated after each incubation step with at least one organic solvent to remove free acid residues and it is initially treated after the first incubation step with boiling water to remove protease residues.

In this context, it has proven advantageous to conduct a first incubation step in a first acid for at least two minutes and a second incubation step in a second acid for at least ten minutes.

Both inorganic and organic acids are usable. The inorganic acids are preferably hydrochloric acid, nitric acid and sulphuric acid. The organic acids are selected from the unsubstituted or substituted C₁-C₆-carboxylic acids, wherein the substituted C₁-C₆-carboxylic acids are preferably selected from the monosubstituted or polysubstituted C₁-C₆-carboxylic acids, particularly preferably from the monohalogenated or polyhalogenated C₁-C₆-carboxylic acids. Of the unsubstituted C₁-C₆-carboxylic acids, acetic acid is most preferred, and the most preferred of the polyhalogenated C₁-C₆-carboxylic acids is TFA.

In a preferred embodiment, both incubation steps are conducted in organic acids, wherein it is particularly preferred to conduct the first incubation step in a first organic acid and the second incubation step in a first second acid.

A particularly preferred embodiment of the method for producing a peptide purification material is characterised in that the first incubation step takes place in a halogenated C₁-C₆-carboxylic acid and the second incubation step takes place in an unsubstituted C₁-C₆-carboxylic acid.

In order to remove free acid radicals, the graphite is treated with at least one organic solvent after both the first and second incubation steps. Such solvents include organic polar aprotic solvents, which are used alone or in mixture with each other or in mixture with water. If mixtures are used, volume ratios of the mixtures are from 1:9 to 9:1. Particularly mixtures with wafer have volume ratios from 1:9 to 9:1, preferably from 1:5 to 5:1, particularly preferably 1:1. A preferred solvent is acetonitrile (ACN).

When the treatment of the graphite has been completed, the graphite is dried. The graphite that has been acidified in this way (pH<7) is usable as peptide purification material. For the purification, it is used in a packed form. The term “packed form” is understood to mean that the material is present as a compacted filling in an apparatus, that is to say slurries are excluded for these purposes.

The method for peptide purification is distinguished by the following steps:
a) bringing an aqueous acidic solution of a peptide with a terminal planar aromatic protecting group into contact with previously acidified graphite in packed form at least once, thus enabling the protecting group to insert itself between the layers of the graphite,
b) washing the graphite containing the peptide-with-protecting group with water, mixtures of water and at least one organic solvent, and with anhydrous organic solvent in successive washing steps,
c) washing the graphite containing the peptide-with-protecting group with an aqueous base,
d) washing the graphite containing the peptide-with-protecting group with a mixture of at least one organic solvent and water to elute the peptide with the protecting group attached from the graphite.

The “aqueous acidic solution of the peptide with terminal planar aromatic protecting group” requires that the protected peptide be cleaved in a mixture of water and an acid, preferably TFA. The mixture may have a volume ratio between 1:9 and 9:1, preferably between 1:5 and 5:1, particularly preferably 1:3 (TFA:water). Anhydrous acid, for example pure TFA or the cleaving solution in which the protected peptide remains on the solid phase after synthesis, prevents any adsorption on the graphite.

In this context, the term “organic solvent” again describes the organic polar aprotic solvents described in the preceding. These may be used alone, or in mixture with each other. Here too, acetonitrile is a preferred solvent.

“Aqueous bases” are selected from the group of inorganic and organic bases, preferably organic bases, ammonia (NH₃)_{(aq)} is used particularly preferably. The aqueous base is preferably used in a concentration between 5 and 50% (V/V), particularly preferably between 20 and 40% (V/V).

The treatment with the aqueous base serves to rebuffer the milieu on the graphite. In this way, the system is prepared for elation of the peptide-with-protecting group. The protected peptide will only be eluted from the graphite if the graphite has bean washed with an aqueous base beforehand. At the same time, the N-terminal protection group is not cleaved. Elution of the peptide with protecting group does not take place in this step, or only to a very limited degree (<5%).

The peptide with protecting group is eluted by washing with a mixture of at least one organic solvent and water. After it has been eluted from the graphite, the peptide with the bound protecting group is obtained with a purity between 70% and 99%, preferably at least 80%, most preferably at least 95%. In this way, the protected peptide is purified from the TFA solution without ether precipitation.

Purification using graphite makes it possible to perform the purification of peptides in parallel processes, which is not possible with HPLC. With HPLC purification, an individual gradient (for example ACN:H₂O or MeOH:H₂O) must be established for each peptide. Moreover, only one peptide can be purified at a time on a preparative HPLC system. Parallel operations enable very many more peptides to be purified in the same period.

For use of the method, the invention accordingly relates to a peptide purification kit, which comprises a device containing previously acidified graphite (pH<7) in packed form. The peptide purification kit preferably comprises a device that contains a graphite-based peptide purification material, that is to say previously acidified graphite, in packed form that has been produced and acidified respectively according to the method described in the preceding. In this context, the “device” particularly comprises any type of column, that is to say normal columns or syringes with a suitable retarding device (for example a frit). An appropriate device is shown schematically in FIG. 1. The graphite is adjusted to a pH<7 (acid) by incubating at least once in at least one organic or inorganic acid for at least one minute as described in the preceding. Preferred embodiments have already been described in the preceding text.

### DESCRIPTION OF THE FIGURES

FIG. 1 Schematic representation of a device for conducting the peptide purification method.

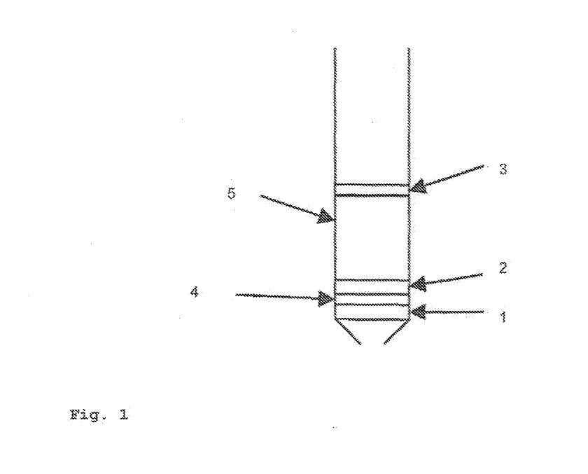

FIG. 2 Shows a control measurement (HPLC-MS) for an Fmoc-protected peptide (Fmoc-LSETKPAV-COOH, also called Fmoc-LSETKPAV-OH) after solid phase synthesis. The protected peptide is disolved in a TFA-water mixture.

FIG. 3 Shows the HPLC-MS chromatogram of the eluate after the Fmoc-protected peptide has been brought into contact with the acidified graphite. The chromatogram shows that the sample has been completely adsorbed, that is to say no more peptide is detectable.

FIG. 4 Shows the HPLC-MS chromatogram of the eluate after the first washing step with water. The chromatogram shows that the sample remains completely adsorbed, that is to say the eluate has no peptide peak.

FIG. 5 Shows the HPLC-MS chromatogram of the eluate after a washing step with pure acetonitrile. The chromatogram shows that the sample remains completely adsorbed, that is to say the eluate has no peptide peak.

FIG. 6 Illustrates the HPLC-MS chromatogram of the eluate after washing with a 30% ammonia solution (volume ratio NH₃:H₂O=3:7). Only very small quantities of free peptide and Fmoc-protected peptide detectable (<5%).

FIG. 7 According to the chromatogram, the eluate that is obtained after the final treatment with 50% ACN (volume ratio ACN:H2O=1:1) includes the peptide with Fmoc group as well as small quantities of the free peptide (without protecting group).

FIG. 8 Shows the HPLC-MS chromatogram after a conventional ether precipitation for the same peptide, but in this case without an Fmoc group, that is LSETKPAV-OH.

### LEGEND

1 First frit
2 Second frit
3 Third frit

### 4 Filter

### 5 Graphite

In the following, the invention will be explained with reference to examples thereof.

### EXAMPLES

### Example 1

### Pretreatment of the Graphite

Approximately 5 g graphite (technical quality) is weighed and introduced into a 20 ml syringe with frit and filter. The graphite is compacted manually by the plunger pressure in the upper portion of the syringe.

This is washed with approximately 10 ml of a TFA-water mixture (volume ratio 1:3; 2.5 ml TFA and 7.5 ml H₂O) and allowed to incubate for 2 minutes. It is then washed 1× with approximately 10 ml boiling water, which kills any proteases on the graphite, and at the same time incubated for 2 minutes.

This is followed by the following washing steps:
1× with approximately 10 ml ACN:H₂O 1:1, 2 minutes incubation,
1× with approximately 10 ml ACN, 2 minutes incubation,
2× with approximately 10 ml 10% acetic acid, with 10 minutes incubation each time, and
1× with approximately 10 ml acetonitrile; 5 minutes incubation.

Then, the graphite is dried.

### Example 2

### Application of the Peptide with Fmoc Group

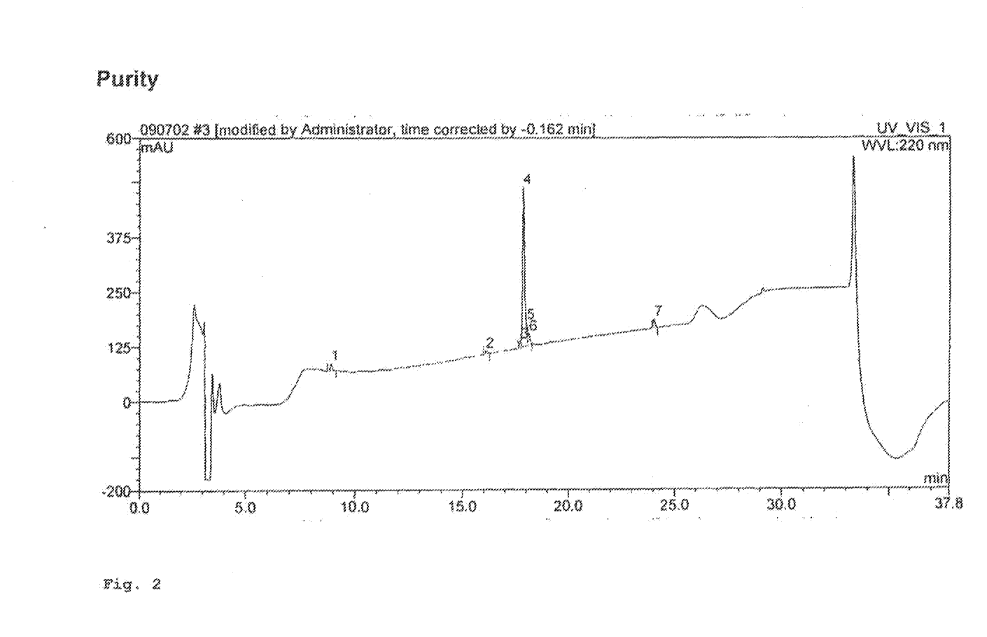

Before the start of the experiment, 1.8 mg of the peptide with Fmoc group (Fmoc-LSETKPAV-COOH) is dissolved in 300 μl TFA und 900 μl water (1:3, v/v) and analysed by HPLC-MS for control purposes (see FIG. 2). This shows that Fmoc peptide (sequence Fmoc-LSETKPAV-COOH) is contained in the solution. The results from FIG. 2 are reproduced in table 1.

TABLE 1

| | Time | Area | Height | | |
|---|---|---|---|---|---|
| No. | UV_VIS_1 | UV_VIS_1 | UV_VIS_1 | Rel. area | |
| UV_VIS_1 | min | mAU * min | mAU | UV_VIS_1 % | |
| 1 | 8.90 | 1.557 | 17.028 | 2.98 | |
| 2 | 16.09 | 0.861 | 7.996 | 1.65 | |
| 3 | 17.70 | 1.179 | 14.441 | 2.26 | |
| 4 | 17.84 | 39.512 | 364.054 | 75.70 | Fmoc peptide |
| 5 | 17.99 | 4.231 | 54.785 | 8.11 | |
| 6 | 18.12 | 2.768 | 27.060 | 5.30 | |
| 7 | 24.02 | 2.090 | 19.808 | 4.00 | |

For the purification, the peptide, in this case Fmoc-LSETKPAV-COOH (=Fmoc-LSETKPAV-OH) with Fmoc group on the N-terminus, is also dissolved in a mixture of TFA and water (volume ratio 1:3) and forced three times through the dry, previously acidified graphite.

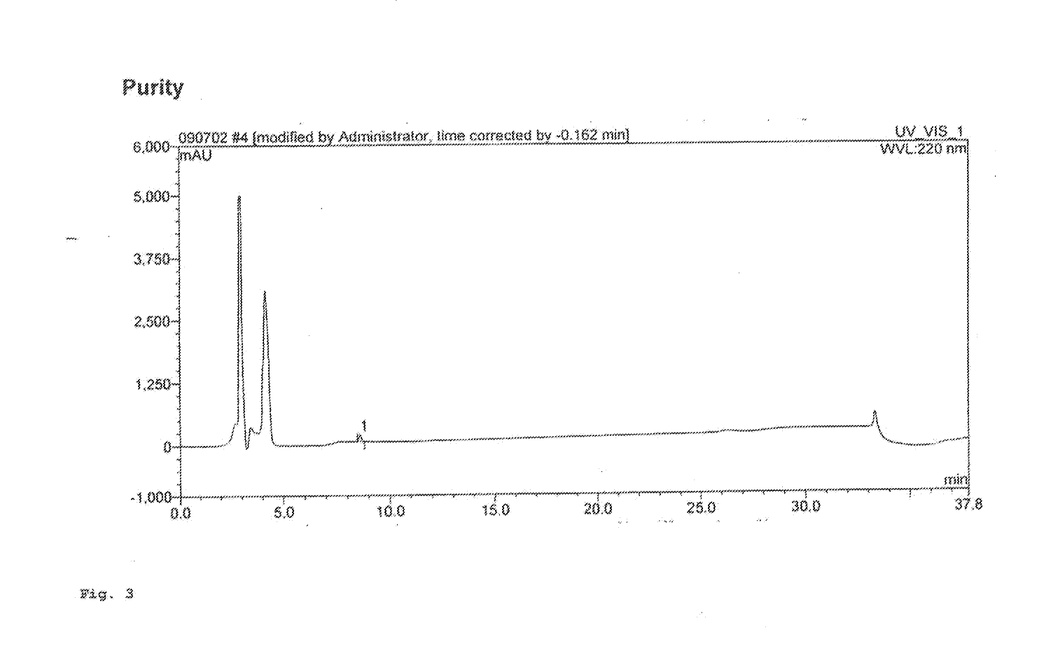

After application of the sample, the eluate is shown by HPLC-MS analysis to have no peptide peak, that is to say the peptide-with-protecting group has been completely adsorbed by the graphite. These results are represented graphically in FIG. 3 and in table 2.

TABLE 2

| | Time | Area | Height | |
|---|---|---|---|---|
| No. | UV_VIS_1 | UV_VIS_1 | UV_VIS_1 | Rel. area |
| UV_VIS_1 | min | mAU * min | mAU | UV_VIS_1 % |
| 1 | 8.59 | 18.056 | 144.630 | 100.00 |

### Example 3

### Washing the Peptide with Fmoc Group on the Graphite

The Fmoc peptide from example 2 that is bound to the graphite is washed as follows:

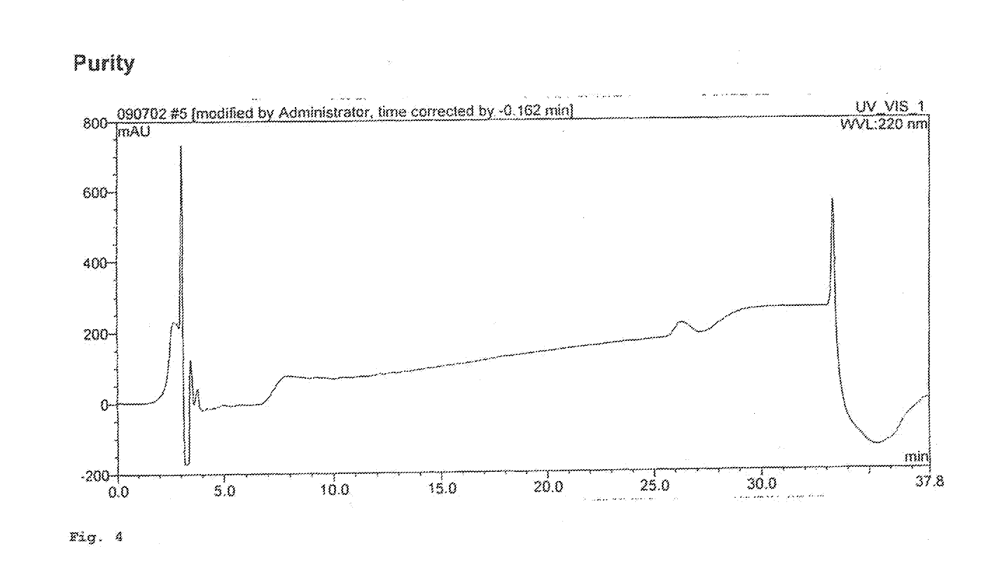

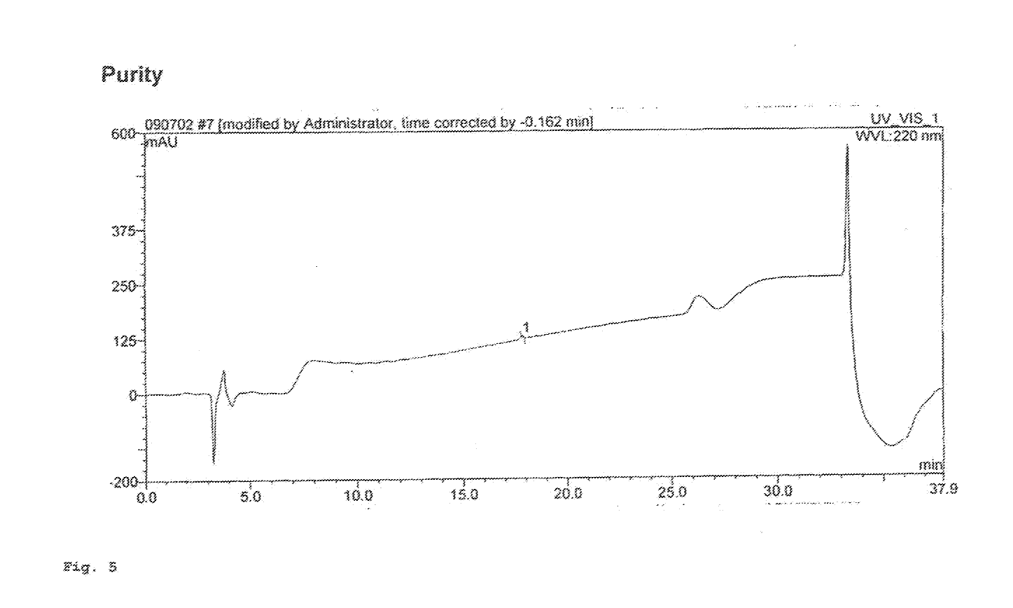

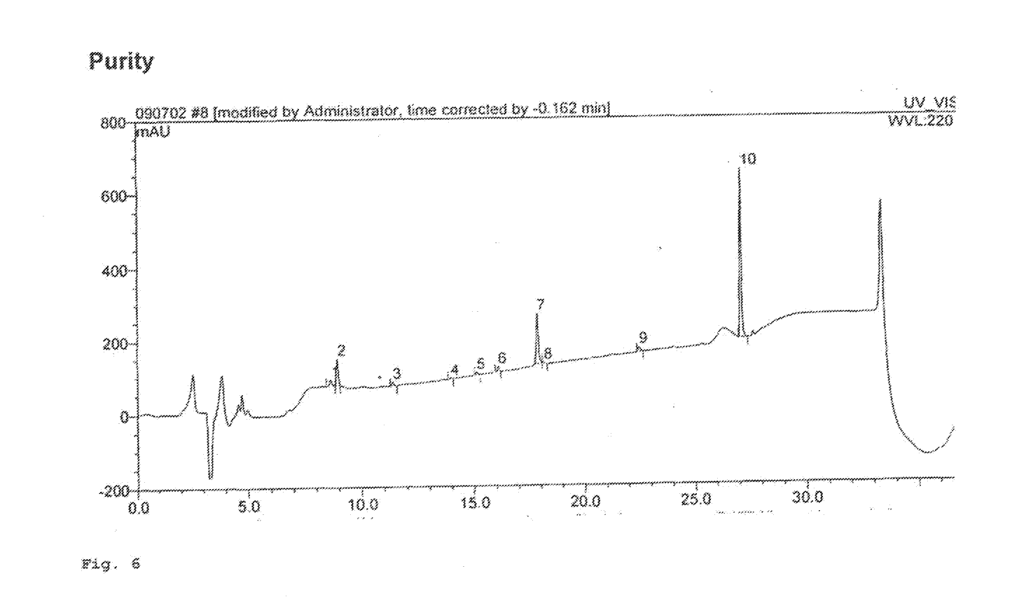

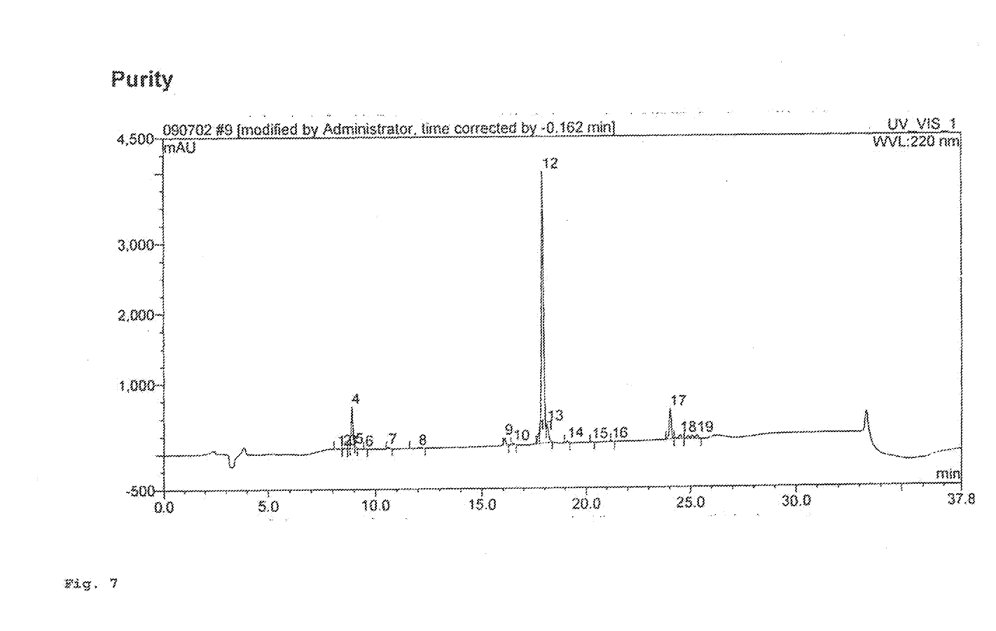

1. 1× with 1.5 ml water (see FIG. 4),
2. 1× with 1.5 ml of a mixture of ACN:H₂O in a volume ratio of 1:1,
3. 1× with 1.5 ml ACN (see FIG. 5);
4. 1× with 1.5 ml 30% NH₃ in water; volume ratio NH₃:H₂O 3:7 (see FIG. 6)
5. 1× with 1.5 ml of a mixture of ACN:H₂O in a volume ratio of 1:1, when the Fmoc peptide is elated out of the graphite again (see FIG. 7).

The eluates from each washing step are analysed using HPLC-MS and presented in FIGS. 4-7. The result from FIG. 5 is also represented in table 3, the result from FIG. 6 in table 4 and the result from FIG. 7 in table 5:

TABLE 3

| | Time | Area | Height | |
|---|---|---|---|---|
| No. | UV_VIS_1 | UV_VIS_1 | UV_VIS_1 | Rel. area |
| UV_VIS_1 | min | mAU * min | mAU | UV_VIS_1 % |
| 1 | 17.84 | 0.614 | 6.545 | 100.00 |

TABLE 4

| | Time | Area | Height | | |
|---|---|---|---|---|---|
| No. | UV_VIS_1 | UV_VIS_1 | UV_VIS_1 | Rel. area | |
| UV_VIS_1 | min | mAU * min | mAU | UV_VIS_1 % | |
| 1 | 8.62 | 1.869 | 16.672 | 2.70 | |
| 2 | 8.90 | 6.351 | 73.704 | 9.18 | Free peptide |
| 3 | 11.35 | 1.128 | 11.530 | 1.63 | |
| 4 | 13.94 | 0.324 | 3.320 | 0.47 | |
| 5 | 15.10 | 0.846 | 8.427 | 1.22 | |
| 6 | 16.09 | 1.378 | 14.565 | 1.99 | |
| 7 | 17.84 | 15.026 | 139.837 | 21.71 | Free peptide |
| 8 | 18.12 | 0.075 | 2.163 | 0.11 | |
| 9 | 22.45 | 1.679 | 13.895 | 2.43 | |
| 10 | 27.04 | 40.524 | 460.444 | 58.56 | |

TABLE 5

| | Time | Area | Height | | |
|---|---|---|---|---|---|
| No. | UV_VIS_1 | UV_VIS_1 | UV_VIS_1 | Rel. area | |
| UV_VIS_1 | min | mAU * min | mAU | UV_VIS_1 % | |
| 1 | 8.20 | 1.068 | 10.320 | 0.16 | |
| 2 | 8.50 | 2.259 | 14.177 | 0.34 | |
| 3 | 8.75 | 0.981 | 12.858 | 0.15 | |
| 4 | 8.89 | 55.472 | 591.596 | 8.31 | Free peptide |
| 5 | 9.04 | 2.456 | 31.448 | 0.37 | |
| 6 | 9.52 | 0.440 | 5.179 | 0.07 | |
| 7 | 10.60 | 2.732 | 28.941 | 0.41 | |
| 8 | 12.00 | 3.049 | 8.097 | 0.46 | |
| 9 | 16.09 | 10.710 | 107.795 | 1.61 | |
| 10 | 16.49 | 1.852 | 22.116 | 0.28 | |
| 11 | 17.69 | 13.716 | 152.854 | 2.06 | |
| 12 | 17.90 | 462.506 | 3873.036 | 69.31 | Fmoc peptide |
| 13 | 18.12 | 31.422 | 270.677 | 4.71 | |
| 14 | 19.09 | 3.070 | 30.774 | 0.46 | |
| 15 | 20.25 | 0.897 | 9.356 | 0.13 | |
| 16 | 21.22 | 0.441 | 5.166 | 0.07 | |
| 17 | 24.02 | 47.518 | 427.910 | 7.12 | |
| 18 | 24.49 | 8.480 | 53.085 | 1.27 | |
| 19 | 25.30 | 18.208 | 51.438 | 2.73 | |

Only the eluate from the last step, that is to say the treatment with ACN-H₂O, shows released Fmoc peptide in significant quantities.

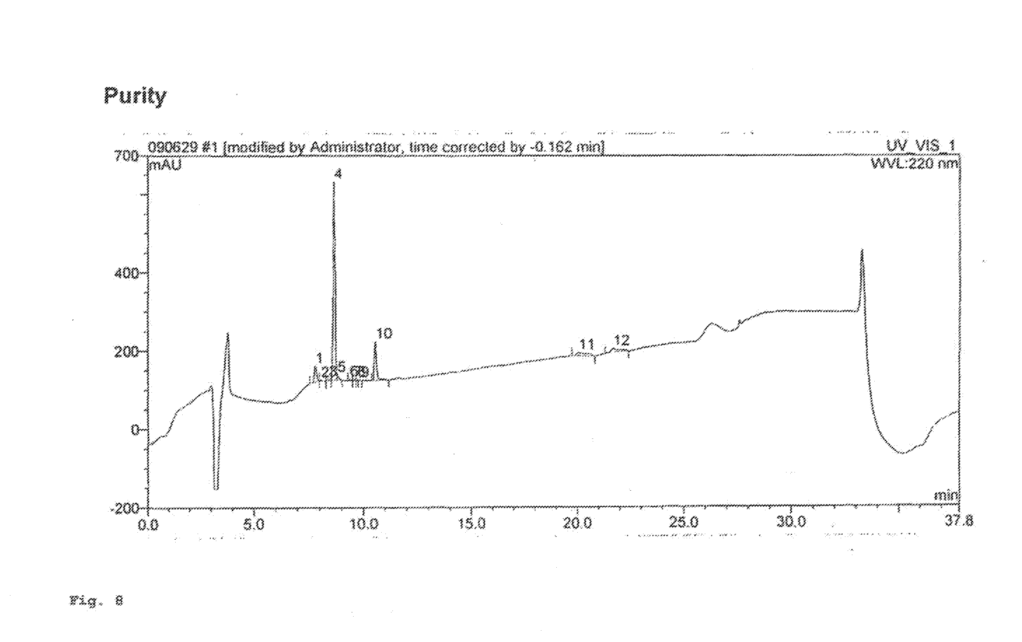

For comparison purposes, the same peptide (without Fmoc group) was also purified by the ether precipitation method. The HPLC-MS analysis yields almost exactly the same result (see FIG. 8), that is to say the purity that is achieved with the method according to the invention is at least equivalent to the purity of the conventional method. The result from FIG. 8 is also reproduced in table 6.

TABLE 6

| | Time | Area | Height | | |
|---|---|---|---|---|---|
| No. | UV_VIS_1 | UV_VIS_1 | UV_VIS_1 | Rel. area | |
| UV_VIS_1 | min | mAU * min | mAU | UV_VIS_1 % | |
| 1 | 7.79 | 5.013 | 40.658 | 6.34 | |
| 2 | 8.05 | 0.065 | 1.463 | 0.08 | |
| 3 | 8.40 | 0.371 | 3.497 | 0.47 | |
| 4 | 8.64 | 51.929 | 507.273 | 65.66 | Free peptide |
| 5 | 8.80 | 1.133 | 13.083 | 1.43 | |
| 6 | 9.39 | 0.323 | 3.638 | 0.41 | |
| 7 | 9.57 | 0.154 | 2.272 | 0.20 | |
| 8 | 9.72 | 0.045 | 0.921 | 0.06 | |
| 9 | 9.87 | 0.015 | 0.277 | 0.02 | |
| 10 | 10.54 | 10.618 | 98.488 | 13.43 | |
| 11 | 20.02 | 4.725 | 7.695 | 5.97 | |
| 12 | 21.65 | 4.697 | 10.718 | 5.94 | |

Subsequent optimisation of the method enabled purities of at least 80%.

1. (canceled)

2. A method for producing a graphite-based peptide purification material, characterized in that graphite is acidified by consecutive incubations in at least two acids for at least two minutes each time, wherein it is treated after each incubation step with at least one organic solvent to remove free acid residues and it is initially treated after the first incubation step with boiling water to remove protease residues.

3. The method for producing a graphite-based peptide purification material according to claim 2, characterized in that a first incubation step takes place in a first acid for at least two minutes and a second incubation step takes place in a second acid for at least ten minutes.

4. The method for producing a graphite-based peptide purification material according to claim 2, characterized in that organic acids are selected from the unsubstituted or substituted C₁-C₆-carboxylic acids, wherein the substituted C₁-C₆-carboxylic acids are preferably selected from the monosubstituted or polysubstituted C₁-C₆-carboxylic acids, particularly preferably from the monohalogenated or polyhalogenated C₁-C₆-carboxylic acids.

5. The method for producing a graphite-based peptide purification material according to claim 3, characterized in that the first incubation step takes place in a halogenated C₁-C₆-carboxylic acid and the second incubation step takes place in an unsubstituted C₁-C₆-carboxylic acid.

6. A method for purifying peptides, wherein the peptide has a terminal planar aromatic protecting group, using graphite in packed form as the peptide purification material, produced by a method according to one or more of claims 2-5.

7. The method for purifying peptides according to claim 6, characterized by the following steps:
e) bringing an aqueous acidic solution of a peptide with a terminal planar aromatic protecting group into contact with previously acidified graphite in packed form at least once, so that the protecting group is able to insert itself between the layers of the graphite
f) washing the graphite containing the peptide-with-protecting group with water, mixtures of water and at least one organic solvent, and with anhydrous organic solvent in successive washing steps
g) washing the graphite containing the peptide-with-protecting group with an aqueous base,
h) washing the graphite containing the peptide-with-protecting group with a mixture of at least one organic solvent and water to elute the peptide with the protecting group attached out of the graphite.

8. The method for purifying peptides according to claim 7, characterized in that the organic solvent is acetonitrile.

9. The method for purifying peptides according to claim 7, characterized in that the aqueous base is NH_{3(aq)} in a concentration between 5 and 50% (V/V), particularly preferably between 20 and 40% (V/V).

10. The method for purifying peptides according to claim 7, wherein the peptide has a fluorenylmethoxycarbonyl group (fmoc) as the terminal planar aromatic protecting group.

11. A peptide purification kit comprising a device containing previously acidified graphite that has been produced according to a method according to any of claims 2 to 5, in packed form.
